(19) United States
(12) Patent Application Publication
(10) Pub. No.: US 2026/0174522 A1
(43) Pub. Date: Jun. 25, 2026

(54) Wearable Augmented Reality Device for Users in a Hospital Environment During Tele-Assisted Procedures and Interaction Process Through Said Device

(71) Applicant: Bernardo DRUMMOND BRAGA, Goiânia-GO (BR)

(72) Inventor: Bernardo DRUMMOND BRAGA, Goiânia-GO (BR)

(21) Appl. No.: 19/426,450

(22) Filed: Dec. 19, 2025

(30) Foreign Application Priority Data
Dec. 19, 2024 (BR) BR102024026822-9
Nov. 19, 2025 (BR) BR102025025418-2

(51) Int. Cl. A61B 90/50; A61B 34/10; A61B 90/00; G02B 27/01; G06F 3/01

(52) U.S. Cl. CPC A61B 90/50; A61B 34/10; A61B 90/361; G02B 27/0101; G02B 27/017; G06F 3/011; A61B 2034/105; A61B 2090/365; A61B 2090/502; G02B 2027/0138; G02B 2027/0141; G02B 2027/0178

(57) ABSTRACT

A wearable augmented reality device for users of a hospital environment in tele-assisted procedures, with at least one lens, at least one connection splitter, at least one processor, network connection, which is configured to integrate users located in different locations during a given procedure; and a process of virtual interaction between a main user and a remote user through said device.

### FIELD OF THE INVENTION

The present invention refers to a wearable augmented reality device to be used by at least one user of a hospital environment in tele-assisted procedures. More specifically, the present invention refers to a virtual reality or augmented reality device configured to integrate users located in different locations during a procedure in a hospital environment with the absence of a video tower. The present invention also refers to a process of virtual interaction through said device.

### DESCRIPTION OF THE STATE OF THE ART

The present application falls within the technical field of augmented reality and virtual reality devices and systems applied and especially aimed at telecare and “teleproctoring” during procedures in a hospital environment. It is an interdisciplinary technological area that combines engineering, computer science, telecommunications, and immersive technologies, aiming to allow interaction between surgeons located in different geographical points during the execution of a specific procedure. Recent advances in connectivity, image processing, optical sensors, and wearable interfaces (wearables) have enabled the expansion of augmented reality applications in clinical contexts.

In conventional procedures, it is common to need face-to-face follow-up by a preceptor user, an expert in a certain technique, to ensure that the surgeon's procedure proceeds without complications or even to resolve such complications, if they occur. These professionals, however, have restricted schedules, which raises costs and makes it difficult for them to be present in multiple locations. In emergency or highly complex situations, the absence of a locally available specialist may compromise the clinical outcome. In these circumstances, the possibility of remote guidance, in real time, would be highly beneficial, but the tools currently available have relevant technical and operational limitations.

Currently, communication between users in different locations is mostly carried out through telephone calls or conventional videoconferencing software. These systems, however, are not able to fully convey the context of that environment, the conditions of the field, and the interactions of the teams. The lack of integration between the physical environment and visual information limits the effectiveness of remote guidance, since critical details of anatomy, movement, and instrumentation cannot be transmitted accurately and simultaneously.

The development of tele-assisted procedures—that is, the performance of procedures at a distance using robotic systems—represented an important milestone in modern medicine. However, the well-known robotic solutions require high-cost equipment, specialized professionals, and very low latency network infrastructure, which limits the widespread adoption thereof. The communication delay between the user's command and the robot's execution remains a critical technical challenge.

In parallel, the concept of teleproctoring has been used in training programs and supervised procedures, allowing specialists to monitor and guide such procedures at a distance. However, this practice still faces important technical obstacles, mainly due to the absence of devices capable of connecting, in an integrated and immersive way, a remote user and a tutored user, with complete sharing of the environment and the field of the procedure. Efficient integration of audio, video, and real-time augmented reality remains a challenge.

Nowadays cell phone cameras, cameras dedicated to digital filming, camera systems of external equipment such as, for example, endoscopes and microscopes, are connected to a computer with internet and through video call software. The procedures are performed with a preceptor or proctor at a distance, who will observe you on his computer. It is necessary to use a microphone and speaker for this preceptor to pass on the guidelines to the main user. This user needs to use a headset, computer speaker or a cell phone. Thus, he often needs to take his eye off the screen of his surgery to make a visual connection with the remote user, at a distance. Gestures can be used by the preceptor to assist the main user in order to show the type of material, type of movement, or demonstrate an important point shared in a video call. At least two cameras are needed, one to show the general environment and the other to show the specific field or location of the procedure. These cameras need an operator (cameraman). In addition to the costs involved, the presence of the cameraman and his equipment occupies space in the hospital environment, hinders the circulation of other technicians, for example, nursing, and can increase the chances of contamination of equipment and the people involved in general.

Therefore, some solutions that seek to apply augmented reality technologies to practices in a hospital environment are known in the state of the art.

Known solutions, therefore, still lack complete integration between capture, transmission, and real-time interaction between geographically separated teams. Most of the known solutions rely on intermediary computers, multiple cameras, and technical operators for equipment adjustment and image capture. Such factors increase cost and operational complexity, in addition to representing additional risk in terms of hygiene and mobility within that environment.

In addition, it is observed that none of the documents mentioned comprehensively contemplates the direct integration between external devices or equipment, for example, of the endoscopic, hemodynamic, and radioscopy types, with wearable autonomous augmented reality systems, nor the possibility of multiple virtual screens in the same field of vision of the main user. Similarly, known solutions do not address the simultaneous visualization of patient exams, preceptor imaging, and field in a single, immersive, manipulable environment.

Thus, the state of the art still brings great limitations, especially regarding operational autonomy, multimodal integration, transmission latency, the need for additional hardware, and high implementation costs. Such limitations indicate the need for more compact, versatile, and affordable solutions, capable of providing immersive support and two-way communication in real time between users located in different locations.

Therefore, there is no wearable augmented reality device in the state of the art for users in a hospital environment during tele-assisted or remote procedures that allows integration between users, patient data, and the hospital environment, with remote collaboration between users in different locations, sharing the entire view of the main user with their preceptor (remote user), including the hospital environment.

More precisely, a wearable augmented reality device for users in a hospital environment in tele-assisted procedures with real-time sharing of a hospital or didactic environment is not seen in the state of the art, compatible with specific 3D printed parts, an audio system integrated with a video system controlled by the main user himself, direct connection to the internet through the device itself and audio and video sharing with the remote user (preceptor), real-time sharing of procedures in a hospital environment, connection between external equipment, for example used in endoscopies with a virtual screen with the absence (without the need) of a video tower.

### OBJECTIVES OF THE INVENTION

The present invention seeks to provide a wearable augmented reality device for users of a hospital environment in tele-assisted procedures.

The present invention seeks to provide a wearable augmented reality device for users in a hospital environment in tele-assisted procedures configured to integrate users located in different locations—physical/geographic locations—during the procedure.

The present invention seeks to provide a process of virtual interaction between a main user and a remote user through a wearable augmented reality device.

### BRIEF DESCRIPTION OF THE INVENTION

The objectives of the present invention are achieved by means of a wearable augmented reality device for users of a hospital environment in tele-assisted procedures, with at least one lens, at least one connection splitter, at least one processor, network connection, and which is connected to external equipment and configured to be worn by a main user and display the images captured by the external equipment in real time on a virtual screen generated on the device itself, further presenting a display of an immersive visualization of a patient's information/data, in a floating and interactive way at least to the main user, through at least one secondary platform.

The objectives of the present invention are further achieved through a process of virtual interaction between a main user and a remote user through a wearable augmented reality device.

The objectives of this invention are further achieved by means of a wearable augmented reality device for users of a hospital environment and a virtual interaction process between a main user and a remote user compatible with each other.

### BRIEF DESCRIPTION OF THE DRAWINGS

The present invention will hereinafter be described in greater detail based on an exemplary embodiment illustrated in the drawings.

FIG. 1 is an example of a main user of a hospital environment with the wearable augmented reality device as proposed by the present invention.

FIG. 2 is an example of the wearable augmented reality device when used in a hospital environment with emphasis on the virtual screen as proposed by the present invention.

FIG. 3 is an example of the wearable augmented reality device when used in a hospital environment with emphasis on the virtual screen as proposed by the present invention.

FIG. 4 is an example of a virtual interaction process through said wearable augmented reality device in a hospital environment with emphasis on the virtual screen and a remote user as proposed by this invention.

FIG. 5 is an example of a main user with the wearable augmented reality device when in use in a hospital environment.

FIG. 6 is a schematic drawing illustrating in an exemplary manner the wearable augmented reality device connected to a connection splitter, to an external equipment, to a remote user, and to virtual equipment according to the teachings of the present invention.

### DETAILED DESCRIPTION OF THE DRAWINGS

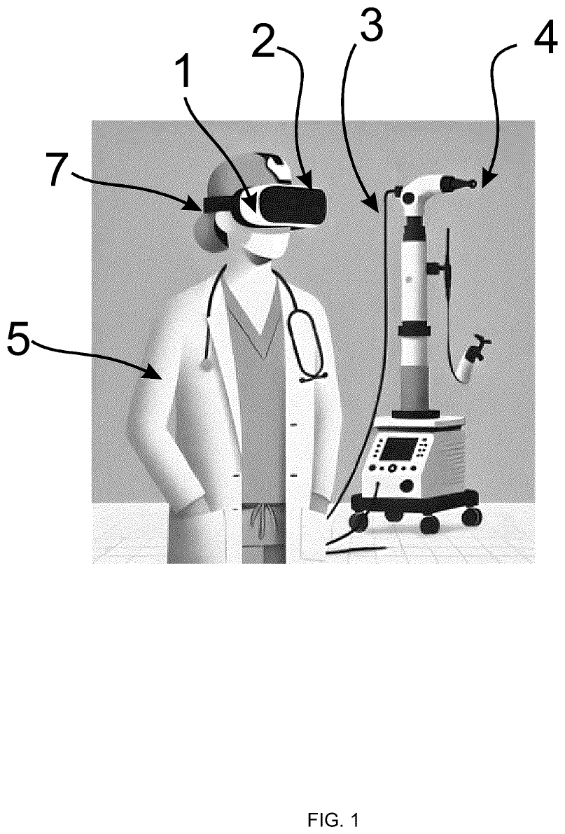

Initial reference is made to FIGS. 1 to 6. In general, the proposed invention falls within the technical field of endoscopic surgeries and telemedicine through a wearable virtual reality or augmented reality device (telesurgery/teleproctoring) for users of a hospital environment in tele-assisted procedures.

The invention object of this patent application is generally related to a wearable augmented reality device 1 for users of a hospital environment in tele-assisted procedures with the absence of an (external) video tower, the device being configured in such a way that it allows the integration of at least two users at a distance.

The proposed technology beneficially allows a user to perform a procedure without the need for an external video tower, screen, and camera capture system. The connection of the external equipment aligned with the invention as proposed takes place directly in the device. Advantageously, this direct connection is much cheaper than the connection through the video tower as proposed by the state of the art, it also optimizes the space of the hospital environment (or operating room) and allows multiple procedures at the same time with better ergonomics for the users.

The proposed solution also allows a remote user—for example, a surgeon in a remote location—to have a visualization of a procedure or act in a hospital environment in real time, on a high-resolution virtual screen 8, showing a visual field of the main user and additional screens that show important information such as, for example, patient exams.

In this aspect, in a configuration it is possible to see the layout of the hospital environment, anesthesia equipment, scopy, and thus provide assistance with real-time technical guidance to the surgeon who performs the technique.

The present invention can be incorporated (connected) to at least one external equipment, such as an endoscope. This configuration provides the user with a clearer view of the entire environment, with higher resolution (e.g. 4K) and adjustable to a preferred size (dimension), improving image quality, ergonomics and efficiency during procedures.

In addition, the proposed invention can be implemented with a direct connection via cable or with a wireless connection through an intermediate computer.

In addition, the object of this invention also includes a process of virtual interaction between a main user 5 and a remote user 6 through a wearable augmented reality device 1.

All the characteristics of the objects of the present invention will be described in detail below.

First, and in reference to FIGS. 1 to 6, the present invention refers to a wearable device of augmented reality 1 for tele-assisted procedures, configured to integrate users of a hospital environment, one of them being a main user 5 and at least one other remote user 6, located in different geographic locations during said procedure.

To this end, device 1 has at least one lens 2, at least one connection splitter 3, at least one processor, network connection, so that the device is preferably of the type “immersive glasses”, “virtual reality glasses”, “3D glasses” or similar, connected to external equipment 4 and configured to be worn by a main user 5 and display the images captured by the external equipment 4 in real time on a virtual screen 8 generated on the device itself 1.

In one embodiment, it is exemplified that device can be at least one among: Meta quest3, Play for Dreams or Apple Vision Pro.

Preferably, the external equipment 4 to which device 1 is connected has at least one camera or a set of cameras and, for example, may be endoscopy, hemodynamic, or radioscopy equipment, that is, equipment that is usually equipped with at least one set of cameras or a microcamera at its end to capture and/or produce images in real time, allowing, for example, visualization of movements of internal structures of a patient's body or radioscopy image capture in which hemodynamic catheters can be observed through the patient's body. An endoscopy procedure may be taken as an example of the preferred configuration or embodiment of this invention throughout this specification.

Alternatively, and except for some adaptations that may be necessary, it is still possible to use the present invention in any procedure in a hospital environment, such as abdominal laparoscopy, thoracic laparoscopy, endoscopic procedures of the gastrointestinal tract, otorhinolaryngology, cerebral ventricular endoscopy, urological procedures, procedures in the digestive system, biliary system, head, neck and skull base procedures, videolaryngoscopy, third ventriculostomy, cardiac and cerebral revascularization, valve replacement, embolization of aneurysms and vascular malformations, endovascular treatments, and several others, mentioned here solely as possible and non-limiting examples.

In any case, external equipment 4 has at least one camera and, alternatively, such a camera may be an integral part of device 1 itself. That is, in a possible configuration, device 1 itself has a set of cameras or at least one camera.

In any case, it is advantageously possible to use device 1 object of this invention without the need for a third party-for example, a cameraman.

The number of cameras is not a limiting characteristic of the proposed invention, that is, it is possible that a smaller or larger number of cameras may be implemented in certain embodiments of the present invention, but still achieving the proposed objectives.

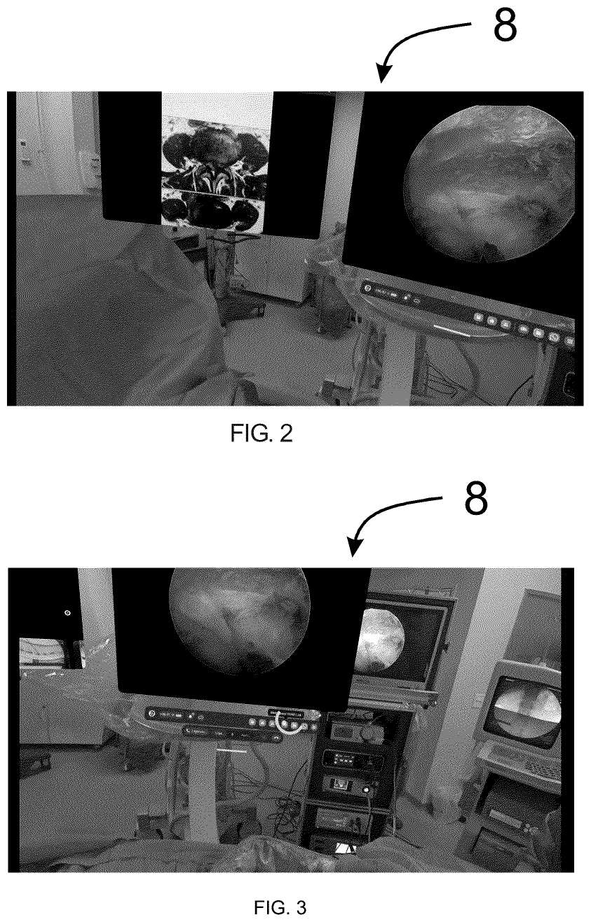

In turn, the virtual screen 8 as shown, for example, in FIGS. 2, 3 and 4, is also configured to display at least one immersive visualization of a patient's information, in a floating and interactive way at least to the main user 5.

In the context of this invention, “virtual” is understood as a screen created in the “digital”—not physical—sphere, so that it does not occupy a physical space in the environment or space. This characteristic is a great advantage over physical screens that take up space in the hospital environment.

The virtual screen 8 allows displaying in this non-physical virtual environment information in various formats such as, for example, visual, textual, video, photos, dynamic images, static images, files, holograms and the like, as well as combinations thereof, in an immersive way to users, related for example to a patient.

In addition, such a virtual screen configuration 8 allows the presentation of multiple smaller screens and the ability to interact with them through secondary platforms that will be further described below—for example, moving the images, changing their sizes with simple “in the air” touches, navigating between screens, etc.

Thus, the virtual screen 8 is configured to display at least one immersive visualization of a patient's information as highlighted especially in FIGS. 2 and 3, in a floating and interactive manner at least to the main user 5, through at least one secondary platform.

For this immersive view on the virtual screen 8, device 1 can be connected to external equipment 4 directly or indirectly, as illustrated schematically in FIG. 6.

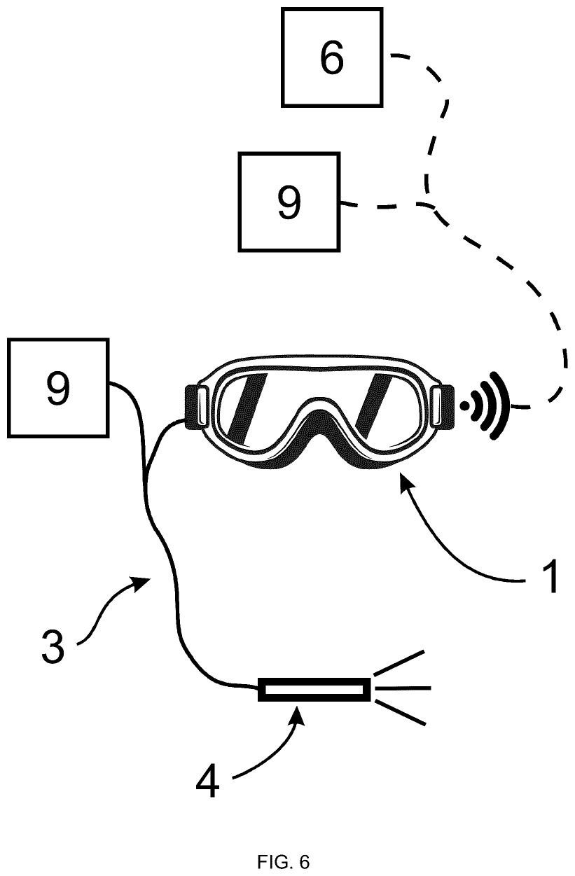

In a direct connection, which is preferred as illustrated mainly in FIGS. 1 and 5, the external equipment 4 (for example, endoscope) is connected directly to the augmented reality device 1 through at least one connection splitter 3 (solid line in FIG. 6) and with the absence of a central tower. In this configuration, therefore, a cable connects a video output from external equipment 4 directly to device 1 without passing through a central tower, thus allowing device 1 to display the captured images in real time without any type of delay or external interference. In this same modality, at least one additional equipment 9 can be connected to device 1, and such connection can therefore be via connection splitter 3 or through a network (dashed line in FIG. 6).

For the use of external equipment such as a disposable endoscopy device that already has a direct USB port, the connection can be made directly to the endoscope without going through a video tower and lighting hardware, as this external equipment 4 already has an adjustable LED light around its camera.

In a preferred configuration, shown in FIG. 1, the connection splitter 3 as used should allow the capture of high-resolution image signals (e.g. Hagibis cables), and may also allow device 1 to be connected to an external power source, thus eliminating the use of batteries.

An alternative is the indirect connection through the video tower (not pictured). Although not a preferred embodiment, it is possible and can also be equally applied in the context of the present invention. The video tower, or endoscopy tower for example, can feature image capture tools in HD or 4K, for example. The indirect connection can be made through an HDMI cable, so that it can be connected to device 1 with a video card with HDMI input and USB output, for example.

In an indirect connection not illustrated, external equipment 4 is indirectly connected to the augmented reality device 1, that is, external equipment 4 is connected to a central tower through at least one cable and the central tower establishes communication (wired or wireless) with the augmented reality device 1, in which the central tower is configured to process a signal received from external equipment 4 and send it in a compatible manner to the augmented reality device 1 for viewing on the virtual screen 8. Here, the cables used must have specific inputs compatible or equivalent to each element.

More specifically, a video output from the central tower is connected to another embedded or non-embedded device (additional equipment 9 such as a computer or processor, for example), configured to transmit the video signal received from external equipment 4 to device 1.

In this configuration, device 1 is configured to process the video signal received through a specific software or program, ensuring compatibility and quality in transmission.

In this embodiment, the central tower may have a light source equipment, an image capture equipment with a preferably high-resolution camera, a television, and a splitter set of HDMI connection inputs that allow the image to be sent to numerous external components, such as a recorder, to the TV and especially to the wearable augmented reality device 1.

It is also a possible alternative that a connection without cables is made, in this case with the help of a computer and a high-speed wireless network to reduce a possible latency (delay) of movements. In any case, the direct connection via cable as described is preferred.

In any case, having a connection established according to one of the possible configurations described, the immersive visualization on the virtual screen 8 may occur through at least one secondary platform, which can interact with each other and at least with the main user 5, in which each secondary platform is digitally configured with a specific function that may be related to the procedure in the hospital environment, the device being able to be configured with a set of functions through each secondary platform.

A first secondary platform is configured as a telephone module, which is configured to allow making voice and/or video calls to at least one remote user 6.

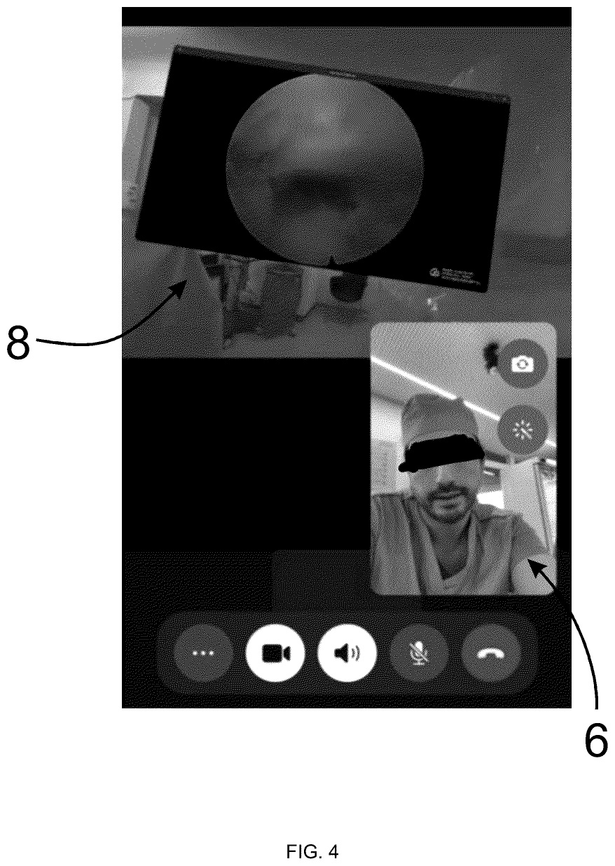

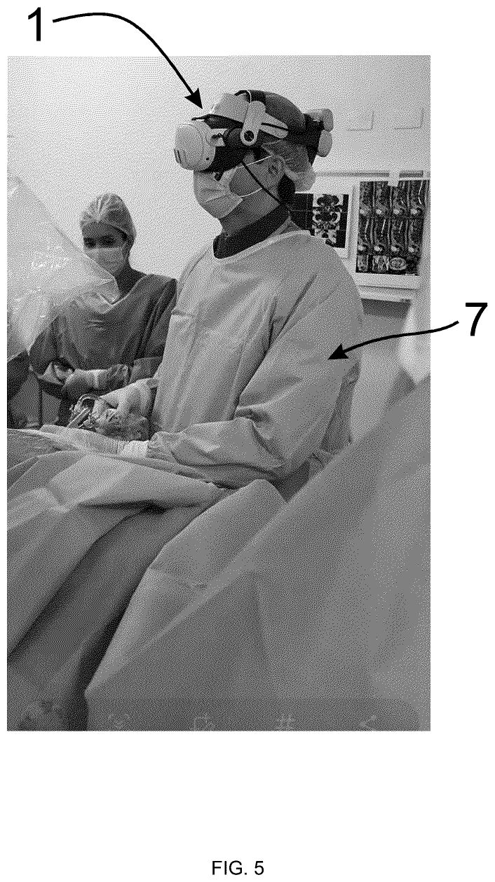

As shown in FIG. 4, the first secondary platform is also configured to allow remote user 6 to receive and view the images displayed on the virtual screen 8 of device 1 in an immersive manner concomitantly with the main user 5. That is, remote user 6 will view on his personal device exactly the same images displayed on the virtual screen 8 of device 1 and that are being viewed in loco by the main user 5, in his field of vision in the hospital environment.

It is exemplified that this first secondary platform can be implemented through software or platforms such as WhatsApp, Zoom, TeamViewer and others.

In addition, a second secondary platform is configured as a data module, configured to store and display information about a patient, in a floating and interactive way, in which such information can be presented in at least one of imaging exams, files, photos, text, graphs, tables and/or combinations thereof.

Such data and information can be obtained through preliminary tests, such as imaging or blood tests, anamnesis, and manual insertion of information in general.

The augmented reality device 1 is configured in such a way that the images captured by it and consequently seen by the main and remote users can be displayed with greater magnification, while also allowing for viewing and interaction on the virtual screen 8 “inside” device 1.

This means that the main user 5 can make certain movements in order to interact with the virtual screen 8 and the content displayed on it through the secondary platforms, as shown in FIG. 5. For example, a magnification of the image can be done using hand movements, amplifying it to the desired size. It is possible to access the patient's exams during the procedure, performing “click” movements on the device's virtual screen 8 and displaying them on the virtual screens while the user works physically/in person in the hospital environment.

As described, the present invention provides that device 1 is connectable in some way to an external electronic device or external (additional) apparatus. Such a feature beneficially allows remote user 6 to participate remotely in the procedure, which implies that. To do so, they can receive a call on their own device—for example, a mobile device, smartphone, tablet, notebook, computer, iPad, augmented reality device, etc.—and interact with the main user remotely, including obtaining (viewing) exactly the same view as the main user (performing surgeon).

This remote interaction provides crucial support to the main user 5 or performing surgeon, allowing the remote user 6 or preceptor to guide the steps of that procedure, adjust equipment, and provide technical recommendations, for example on the manipulation of the endoscope and surgical instruments. This assistance can occur at specific times or throughout the duration of the procedure.

As illustrated in FIG. 6, in addition to the connection with remote user 6, device 1 may also connect to at least one or more additional equipment 9 that may be in loco—by direct connection via connection splitter 3 or additional cable—or remote—by indirect connection via network—, which may also be a television, screen, projector, hologram display and the like in addition to the examples already mentioned above.

Given the characteristics previously detailed, it is also noted that device 1 is compatible with a head support 7 to be placed on a user's head and position said device 1 in front of the eyes, allowing the main user 5 to wear such device 1.

It is also important to mention that device 1 as described and claimed here may be compatible with robotic devices for surgical procedures such as, for example, the Da Vinci Intuitive Labs robot.

In addition to the wearable augmented reality device 1 for users of a hospital environment in tele-assisted procedures, a virtual interaction process between a main user 5 and a remote user 6 through said device 1 is also the object of this invention, so that device 1 and the process are compatible with each other. Thus, except for adaptations, the characteristics of the device already described also apply to the present case.

In general terms, the process is configured to be implemented through device 1 and comprises a series of steps that will be detailed below.

One step of the proposed process comprises connecting device 1 to an external network, for example the internet, and to an external equipment 4, either directly—through at least one cable and with the absence of a central tower, which is a preferred configuration—or indirectly—in which the external equipment 4 is connected to a central tower through at least one cable and the central tower establishes wireless communication with device 1, which is an alternative configuration—as already described.

One step of the proposed process comprises displaying the images captured by the external equipment 4 in real time on a virtual screen 8 generated on device 1 itself. Such a display can be made interactively with the main user 5 and can also be replicated to a remote user 6.

Thus, it is also a step in the proposed process to display at least one immersive visualization of a patient's information, in a floating and interactive way at least to the main user 5, through at least one secondary platform, which has already been previously described, in which such platforms act in specific stages of the proposed process.

A step of making voice and/or video calls to at least one remote user 6 through a first secondary platform configured as a telephone module, in which the first secondary platform is further configured to allow the remote user 6 to receive and view the images displayed on the virtual screen 8 of device 1 in an immersive manner concomitantly with the main user 5. Here, high-resolution patient information can also be displayed, such as exams.

In one embodiment, the process also includes a step of performing a recording (storage) of at least the virtual screen 8 and/or the image obtained by the external equipment 4 through the second secondary platform.

An additional step of the proposed process also includes processing, through a processor, a signal received from the external equipment 4 and sending it in a compatible way to the augmented reality device 1 for display on its virtual screen 8.

It is also a step in the proposed method to attach device 1 to a head support 7, so that said device 1 can be worn by a main user 5 such as glasses.

The process described is therefore compatible with the characteristics of device 1 also described here and, in one configuration, comprise similar characteristics that are compatible to each other and achieve the advantages detailed herein.

In addition, a possible embodiment of the present invention relates to a teaching environment, in which the proposed invention can be used for training a certain procedure without it actually taking place in a real human patient, that is, before it happens and outside a real hospital environment, but rather in a didactic environment.

To this end, device 1 and external equipment 4 are connected to a specific simulator, which in turn reproduces by means of 3D printing pieces identical to the anatomy of a certain structure of the human body to be studied and which may in the future be the object of a procedure in a hospital environment. For example, a 3D print of the patient's anatomy is made for study before the actual procedure is performed.

In this embodiment, there is a preliminary capture of patient images, for example, through tomography and magnetic resonance imaging, which are later converted into a format compatible with 3D printing software such as STL. A printer then prints a three-dimensional physical piece of a certain anatomical region of the patient that will be submitted to the procedure. The physical model created is made in order to reproduce the exact dimensions of that patient, the model being taken to a simulation environment with specific structures for this type of study-for example, a laboratory with surgical forceps similar to those used in a hospital environment.

In this way, the invention can be embodied as a simulator or training platform, used for example for the purpose of courses, improvement of techniques and the like. In this case, remote user 6 can be a tutor and main user 5 a student, for example.

Finally, it is understood that the description of some elements and characteristics of the system and method proposed in this invention should not be considered as limiting characteristics of the invention.

For example, the teachings of this invention can also be applied not only to medical professional users of a hospital environment, but also to a context of learning, tutoring, mentoring, training and the like.

Also, the teachings of this invention can be applied to more than one procedure in a hospital environment.

Some advantages of the invention proposed herein are summarized below.

The invention, as described and defined in the claims, can be beneficially used in real procedures as well as in simulators made in 3D printing for tutoring and learning techniques with a tutor at a distance.

The invention as described and defined in the claims makes it possible to dispense with an exclusive user to carry out filming—cameraman—also dispensing with the eventual implementation of a complex set of cables, wires and an external video tower to make a transmission. In other words, the present invention makes it possible to use the Wearable Augmented Reality Device 1 for users of a hospital environment in tele-assisted procedures without the presence of a cameraman and without the use of a video tower.

The invention as described and defined in the claims allows you to have virtual screens that do not occupy a physical space.

The invention as described and defined in the claims allows a main user 5 to receive essential support from at least one remote user 6 to improve their proficiency in new techniques, broadening their understanding of anatomy, promoting precise and delicate movements with accuracy.

The invention as described and defined in the claims contributes significantly to patient safety and well-being since the remote user 6 can assist the main user 5 (executor) at a critical moment, minimizing costs, chances of errors and fatalities.

The invention as described and defined in the claims allows displaying high-resolution images directly on the virtual screen 8 of device 1, ensuring maximum visual quality.

The invention as described and defined in the claims allows quick visualization of important patient information, such as exams, minimizing the chances of errors.

The invention as described and defined in the claims allows for a faster and safer evolution of users for the acquisition and diffusion of new techniques.

The invention as described and defined in the claims significantly reduces costs related to certain procedures, since it is not necessary for the remote user 6 (expert) to be in person at the same location as the main user 5.

The invention as described and defined in the claims allows the establishment of a connection of professional help for critical situations, education, mentoring, and also for the acquisition of new techniques to be implemented in a hospital environment.

Having described a preferred embodiment, it should be understood that the scope of the present invention encompasses other possible variations, being limited solely by the content of the appended claims, potential equivalents included herein.

### NUMERICAL REFERENCES

Wearable Augmented Reality Device 1
Lens 2
Connection splitter 3
External equipment 4
Main user 5
Remote User 6
Head support 7
Virtual screen 8
Additional equipment 9.

1. A wearable augmented reality device for users of a hospital environment in tele-assisted procedures, with at least one lens, at least one connection splitter, at least one processor and network connection, wherein the wearable augmented reality device is connected to an external equipment and configured to be worn by a main user and display images captured by the external equipment in real time on a virtual screen generated on the device itself,
wherein the connection to the external equipment occurs directly with the absence of a video tower,
wherein the virtual screen is also configured to display at least one immersive view through at least one secondary platform,
wherein the secondary platforms are capable of interacting with each other and with the main user,
wherein each of the at least one secondary platform is digitally configured with a specific function that can be related to the procedures,
wherein the device is configured with a set of functions through each of the at least one secondary platform, and
wherein at least
a first secondary platform is configured as a telephone module, and
a second secondary platform is configured as a data module.

2. The wearable augmented reality device, according to claim 1, wherein the external equipment has at least one camera or microcamera to capture and/or produce images in real time.

3. The wearable augmented reality device, according to claim 1, wherein the external equipment is connected directly to the wearable augmented reality device through at least one connection splitter, wherein the connection splitter is configured to allow the transmission of the images captured by the external equipment and which is viewed by the main user through the device, wherein the images captured by the external equipment is within a field of view of the main user and is transmitted to at least one among a remote user, a physical screen and/or additional equipment.

4. The wearable augmented reality device, according to claim 1, wherein the first secondary platform is configured as a telephone module, which is configured to allow voice and/or video calls to at least one remote user, wherein the first secondary platform is further configured to allow the remote user to receive and view the images displayed on the virtual screen of the device in an immersive manner concurrently with the main user.

5. The wearable augmented reality device, according to claim 1, wherein the second secondary platform is configured as a data module, configured to store and display information of a patient, in a floating and interactive manner, in which such information is presented in at least one of imaging exams, files, photos, text, graphs, tables and/or combinations thereof.

6. The wearable augmented reality device, according to claim 1, wherein the device is connected to at least one external electronic device.

7. The wearable augmented reality device, according to claim 1, wherein the device is compatible with a head support to be placed on a head of user and position the device in front of the eyes, allowing the main user to wear the device.

8. The wearable augmented reality device, according to claim 1, wherein the device is connectable to an internet network.

9. The wearable augmented reality device, according to claim 1, wherein the device is configured as immersive glasses.

10. A process of virtual interaction between a main user and a remote user through a wearable augmented reality device as defined in claim 1, comprising at least the steps of:
connecting the device to an external equipment;
displaying images captured by the external equipment in real time on a virtual screen generated on the device itself;
displaying at least one immersive visualization of information of a patient, in a floating and interactive manner at least to the main user, through at least one secondary platform.

11. The process of virtual interaction through a wearable augmented reality device, according to claim 10, wherein the step of connecting the device to an external equipment is carried out directly through at least one connection splitter and in the absence of a central tower.

12. The process of virtual interaction through a wearable augmented reality device, according to claim 10, further comprising a step of processing a signal received from the external equipment and sending the signal in a compatible manner to the augmented reality device.

13. The process of virtual interaction through a wearable augmented reality device, according to claim 10, further comprising a step of making voice and/or video calls to at least one remote user through a first secondary platform configured as a telephone module, wherein the first secondary platform is further configured to allow the remote user to receive and view the images displayed on the virtual screen of the device in an immersive manner concomitantly with the main user.

14. The process of virtual interaction through a wearable augmented reality device, according to claim 10, further comprising a step of storing and displaying information of a patient in high resolution, in a floating and interactive way, in which such information may be related at least to imaging exams and be presented in at least one among images, files, photos, text, graphs, tables and/or combinations thereof.

15. The process of virtual interaction through a wearable augmented reality device, according to claim 10, further comprising a step of making a recording of at least the virtual screen and/or the image obtained by the external equipment through the second secondary platform.

16. The process of virtual interaction through a wearable augmented reality device, according to claim 10, further comprising a step of connecting the device to at least one external electronic device.

17. The process of virtual interaction through a wearable augmented reality device, according to claim 10, further comprising a step of attaching the device to a head support.

18. The process of virtual interaction through a wearable augmented reality device, according to claim 10, further comprising a step of connecting the device to an internet network.

19. The process of virtual interaction through a wearable augmented reality device, according to claim 10, wherein the process is compatible with the device.
